(19) United States
(12) Patent Application Publication
(10) Pub. No.: US 2005/0148792 A1
(43) Pub. Date: Jul. 7, 2005

(54) Process for the preparation of gabapentin

(76) Inventor: Damodaragounder Gopal, Mayfield Heights, OH (US)

Correspondence Address: Ricerca Biosciences, LLC, 7528 Auburn Road, Concord, OH, 44077, US

(21) Appl. No.: 10/749,199

(22) Filed: Jan. 2, 2004

(51) Int. Cl. C07C 227/12

(52) U.S. Cl. 562/503

(57) ABSTRACT

A simple, efficient, environmentally improved and economical process for preparing gabapentin, involving enamine alkylation as the key step is disclosed.

### FIELD OF THE INVENTION

The present invention relates to a new process for preparing gabapentin.

### BACKGROUND OF THE INVENTION

Gabapentin (1-aminomethyl-1-cyclohexaneacetic acid) is an orally active central nervous system (CNS) drug whose activity is akin to that of the neurotransmitter, γ-aminobutyric acid (GABA). It crosses the blood-brain barrier, and is widely used for the treatment of epilepsy, faintness, hypokinesia, anxiety disorders, cranial trauma, and pain [Satzinger, G. et al. U.S. Pat. No. 4,024,175; 1977]. Due to its low toxicity and lack of known metabolites, it can be prescribed in high doses (c.a. 2 g/day) [Schmidt, B. In Antiepileptic Drugs. Levy, R. H. et. al. (Eds.), Raven Press: New York, 1989, pp. 925-935; Goa, K. L., Sorkin, E. M. Drugs 1993, 46, 409-427; Pande, A. C. et al. J. Clin. Pyschopharmacol. 1999, 19, 341-345]. Since its discovery in 1975, gabapentin has been synthesized by many different methods described in the following patents, which are incorporated by reference in their entireties: Satzinger, G. et al. U.S. Pat. No. 4,024,175; 1977; Mettler, H. P. et al. U.S. Pat. No. 4,958,044; 1990; Geible, W. et al. U.S. Pat. No. 5,091,567; 1992; Mettler, H. P. et al. U.S. Pat. No. 5,130,455; 1992; Jennings, R. A. et al. U.S. Pat. No. 5,132,451; 1992; Bryans, J. S. et al. PCT WO 99/14184; 1999.

The key requirements for an ideal chemical manufacturing process are: (a) high yield, (b) atom economy, (c) minimal number of steps; (d) convergence, (e) minimal use or avoidance of toxic and/or flammable solvents and reagents, (f) low cost of raw materials, (g) avoidance of extreme temperature and pressure conditions, (h) avoidance of explosive intermediates, (i) use of environmentally compatible solvents, (j) recoverable reagents and intermediates, and (k) minimal waste disposal quantities. Although it is rare for any chemical process to satisfy every criteria listed above, all the prior art methods for manufacturing gabapentin and its analogs are deficient in many important aspects, including, but not limited to the use of cryogenic conditions, the use of toxic reagents such as cyanide, moderate yields, the use of potentially explosive intermediates such as azides and nitromethane, and the use of flammable alkali metal. Thus, there continues to exist need for an efficient process for the preparation of gabapentin that avoids many of the aforementioned drawbacks. Accordingly, the object of this invention is to disclose novel and economical processes for the preparation of gabapentin that are safer and environmentally more benign than those used at this time.

### SUMMARY OF THE INVENTION

The present invention relates to a novel method for the preparation of gabapentin (Formula 1) from the key intermediate enamine of Formula 2, 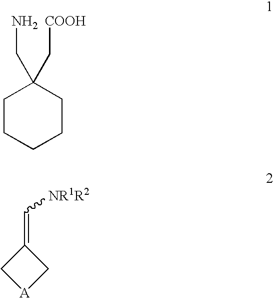
wherein A is propylene or propenylene and R¹ and R² may be chiral or achiral and are independently selected from the group consisting of hydrogen, C₁-C₁₀ alkyl, C₃-C₁₀ cycloalkyl, C₅-C₁₅ aryl (unsubstituted or substituted with halogen, trihalomethyl, cyano or nitro), C₁-C₁₀ alkoxycarbonyl, hydroxyl and C₁-C₁₀ alkoxyl.

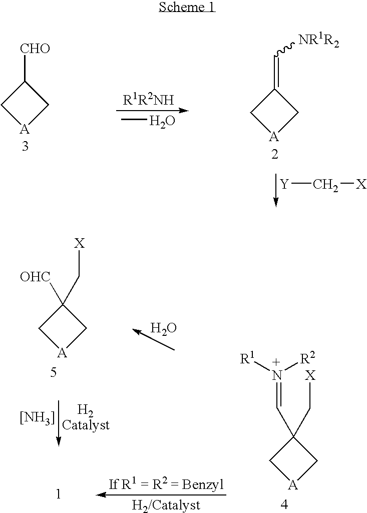

The first step of the process of the present invention (Scheme 1) involves the preparation of enamines 2 by condensing carboxaldehydes 3 with any suitable secondary amines. The second step involves the preparation of iminium salts 4 by the alkylation of said enamines with various alkylating agents, Y—CH₂—X, wherein Y is a leaving group selected from halogen, C₁-C₁₀ alkane sulfonate, and C₅-C₁₀ arene sulfonate and X is selected from the group consisting of-CN, —CO₂M, —CO₂R₃ and —CONR₄R₅, with R₃ to R₅ being independently selected from the group consisting of hydrogen, cyanoethyl, alkyl cycloalkyl, aryl unsubstituted or substituted with electron withdrawing or electron donating groups; arylalkyl unsubstituted or substituted with electron withdrawing or electron donating groups, and M is selected from the group consisting of lithium, sodium, potassium, calcium, magnesium, trialkylammonium and tetralkylammonium. The third step involves the conversion of the iminium salts 4 to gabapentin 1 by (a) hydrolysis to an aldehyde 5 followed by reduction or (b) direct reduction to gabapentin, if R¹ and R² are benzyl groups.

### DETAILED DESCRIPTION OF THE INVENTION

The present invention provides a convenient, safe, and environmentally improved process for the preparation of gabapentin, employing common steps to produce an iminium salt, followed by alternate steps to yield the final product. Where the salt is hydrolyzed to the aldehyde 5, Scheme 2 shows alternate methods to reach the final product. 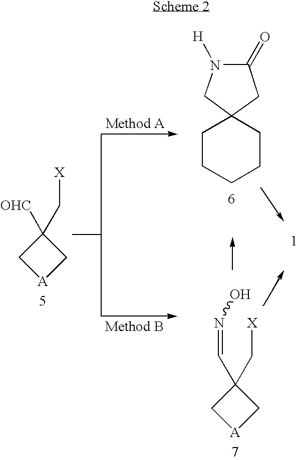
In Method A, the aldehyde 5 is converted to Product 1 by reductive amination using ammonia and hydrogen. If X is a benzyl ester, acid or a salt, then the reductive amination will directly yield gabapentin. If X is other than a benzyl ester group, then the reaction proceeds through the lactam 6 to give gabapentin. In either case, the lactam is converted to gabapentin by reductive amination. In Method B, the aldehyde 5 is first converted to the oxime 7, and then reduced to gabapentin directly or through the intermediate lactam as described earlier.

The starting materials, aldehydes 3 and the secondary amines, are either available commercially or can be readily prepared by methods well known in the art. For example, cycloalkane carboxaldehydes can be prepared by hydroformylation reaction of their corresponding olefins, and 3-cycloalkene carboxaldehydes can be prepared via Diels-Alder reaction of acrolein and butadiene. The condensation of the aldehydes 3 with secondary amines to give enamines 2 is typically carried out by using a catalytic amount of acid or an acid resin or it can be performed without catalyst under azeotropic removal of water. The removal of water can be also achieved by using potassium carbonate as a dehydrating agent. Typical solvents for this reaction include, but are not limited to, toluene, tetrahydrofuran (THF), heptane, dimethoxy ethane, acetonitrile, methylene chloride, ethylene dichloride, and the like. The choice of secondary amines can vary widely depending on availability, recoverability and atom economy, and on the particular route of making the final product. The secondary amines employed in the present invention may be chiral or achiral, alkyl or arylalkyl amines and include, but are not limited to, diisopropylamine, diisobutylamine, t-butylbenzylamine, dibenzylamine, pyrrolidine, piperdine, morpholine, R- or S-proline, R- or S-2-pyrrolidine methanol, R- or S-2-methoxmethylpyrrolidine, N,α-dimethylbenzyl-amine, RR- or SS-2,5-dimethylpyrrolidine, and the like.

The C-alkylation of enamines 2 with various saturated, unsaturated, substituted, or unsubstituted halogenated hydrocarbons, halogenated acetals and ketals, mesylates, tosylates, epoxides, Michael acceptors, and the like represents the novel, and essential step in the process of the present invention. The enamine alkylation method (the Stork reaction) to form carbon-carbon bonds has been widely used to prepare various other natural and synthetic products [House, H. O. The Formation and Alkylation of Enamines. In Modern Synthetic Reactions, W. A. Benjamin: New York, 1972, pp. 570-595]. The alkylation is typically, but not always, carried out in polar aprotic solvents including dimethylformamide (DMF), acetonitrile, dimethylsulfoxide (DMSO), dimethoxy ethane, and the like. If the alkylation is carried out with haloesters, then an additional hydrolysis or hydrogenolysis step will be required to obtain the corresponding acids.

The iminium salts 4 can either be isolated or hydrolyzed in situ to the aldehydes 5. The iminium salts can also be reacted with various nucleophiles such as amines, thiols, thiolates, alcohols, alkoxides, cyanide, enolate anions, and the like. For example, the reaction of 4 with hydroxylamine will produce the oxime 7 directly. The hydrolysis can be performed with or without the use of acids or bases. Transformation of the carboxaldehydes 5, which are key intermediates in the process, to the final product is accomplished by one of the two methods described below.

Method A (Scheme 3). This method involves the preparation of gabapentin by direct reductive amination of the formylesters 8 or formylacids 9 with ammonia and H₂/catalyst. The catalyst employed in this reaction may vary widely depending on the cost, safety, 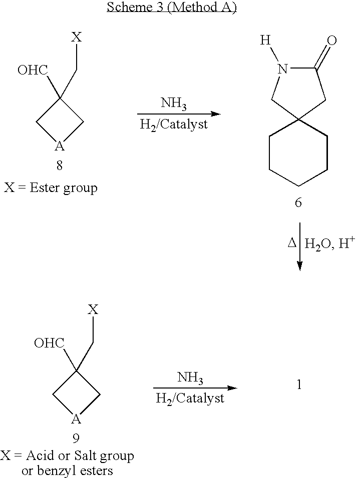
availability, and compatibility with the other functional groups in the molecule. These include, but are not limited to, platinum, palladium, nickel, rhodium, and the like. The reductive amination can also proceed in two stages wherein a geminal aminoalcohol or aminoether is first formed with ammonia and is then converted by hydrogenolysis. Alternatively, reductive amination can be carried out by using ammonium formate and a catalyst such as Pt or Pd (transfer hydrogenation condition), or with a metal hydride agent such as sodium cyanoborohydride or sodium tiacetoxyborohydride. In a preferred embodiment, the method pertains to reductive amination of the ester 8. In another preferred embodiment, the method pertains to reductive amination of the acid 9.

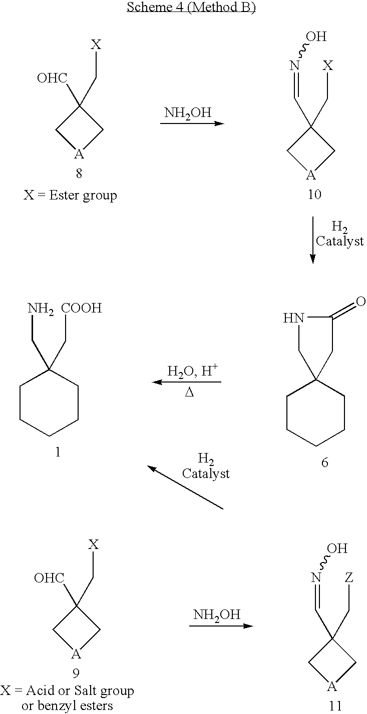

Method B (Scheme 4). This method involves the preparation of gabapentin from the carboxaldehyde 5 in two or three steps. That is, formation of oximes 10 or 11, reduction with H₂/catalyst or with metal hydrides and hydrolysis, if necessary. In this method, hydroxylamine can be replaced with O-alkyl, O-aryl, or O-arylalkyl hydoxylamines to give the corresponding O-alkyloximes, which can also be reduced by the same procedures to produce gabapentin. In a preferred embodiment, the method pertains to the reduction of the oxime ester 10, following which the resulting bicyclic lactan intermediate 6 is converted to gabapentin by hydrolysis. In another preferred embodiment, the method pertains to the direct conversion to gabapentin by reduction of the oxime acid 11.

The methods of the present invention offer many advantages over the existing processes. These include: (a) atom efficiency, (b) recoverable reagents, (c) inexpensive starting materials, (d) flexible synthetic schemes that can accommodate more than one raw material or intermediate, (d) avoidance of explosive or toxic intermediates and reagents, and (e) avoidance of cryogenic conditions. For example, the use of a high boiling secondary amine such as diisobutylamine may enable the recovery of this reagent. High efficiency in preparing gabapentin can be achieved by alkylating the enamine 2 with bromoacetic acid or the carboxylate salt of bromoacetic acid, followed by hydrolysis and reductive amination. The methods of the present invention are also safer because they do not employ toxic reagents, such as cyanide, or explosive intermediates, such as azide.

The following examples describe the preferred embodiments of the invention and are not purported to limit the scope of the invention. It is intended that the specification, together with the following examples, be considered exemplary only, with the scope and spirit of the invention being indicated by the claims that follow these examples. Other embodiments within the scope of claims herein will be apparent to one skilled in the art from consideration of the specification or practice of the invention as described herein.

### EXAMPLE 1

### Synthesis of Gabapentin by Method A

Step 1: Preparation of ethyl (1-formylcyclohexyl)acetate. A mixture of cyclohexanecarboxaldehyde (22.4 g) and diisobutyl amine (28 g) in toluene (150 mL) was heated under reflux with azeotropic removal of about 3.5 mL of water. Toluene was removed by evaporation in vacuo and the residue was treated with 200 mL of acetonitrile. Ethyl bromoacetate (40 g) was added and the reaction was heated under reflux for 16 hours. A solution of acetic acid (20 mL) and sodium acetate (10 g) in 100 mL of water was added to the reaction mixture, and the solution was heated under reflux for one hour. The solvent was removed by distillation, and the product, ethyl (1-formylcyclohexyl)acetate, was extracted with ether. Yield 29.5 g. ¹H NMR (CDCl₃) d 9.63 (s, 1H), 4.11 (q, 2H, J=7.1 Hz), 2.54 (s, 2H), 1.90-1.80 (m, 2H), 1.60-1.35 (m, 8H), 1.24 (t, 3H, J=7.1 Hz). ¹³C NMR (CDCl₃) d 205.9, 171.1, 60.9, 47.9, 41.5, 31.1, 25.6, 22.2, 14.4.

Step 2: Preparation of gabapentin. A sample of 3 g of ethyl (1-formylcyclohexyl)acetate from Step 1 was dissolved in 50 mL of ethanol saturated with ammonia. The mixture was hydrogenated at 50 psi with 10% Pd—C (0.6 g) for 4 hours. The reaction mixture was filtered and the filtrate evaporated in vacuo to give 2.5 g of the intermediate lactam. A mixture of the lactam (2.3 g) and concentrated hydrochloric acid (50 mL) was heated under reflux for 20 hours. Water and excess HCl were removed by evaporation in vacuo to give 1.25 g of gabapentin hydrochloride, which was purified by crystallization from butanol/ether to give 270 mg of pure gabapentin hydrochloride. ¹H NMR (DMSOd₆) d 8.2 (bs,), 2.90 (s, 2H), 2.44 (s, 2H), 1.50-1.30 (m, 10H). ¹³C NMR (DMSOd₆) d 173.5, 45.8, 39.1, 35.3, 33.1, 25.9, 21.4.

### EXAMPLE 2

### Synthesis of Gabapentin by Method A

Step 1: Preparation of (1-formylcyclohexyl)acetic acid. Ethyl (1-formyl-cyclohexyl)acetate (5 g) from Example 1, Step 1 was taken up in ethanol (100 mL, 190 proof) containing 1.62 g of potassium hydroxide. Water (4 mL) was added and the reaction was stirred under inert atmosphere for 16 hours. The ethanol was removed by evaporation and 50 mL of water was added. The aqueous layer was washed with ether. Acidification of the aqueous layer with hydrochloric acid and extraction of the product with ether gave 4.0 g of the acid.

Step 2: Preparation of gabapentin. (1-formylcyclohexyl)acetic acid (6.8 g) was dissolved in 250 mL of ethanol saturated with ammonia. The mixture was hydrogenated at 50 psi with 10% Pd—C (0.6 g) for 4 hours. The reaction mixture was filtered and evaporated in vacuo to give crude gabapentin, which was then purified by crystallization from ethanol/ether.

### EXAMPLE 3

### Synthesis of Gabapentin by Method A

Step 1: Preparation of the lactam, 2-aza-spiro[4,5]decan-3-one. The ethyl (1-formylcyclohexyl)acetate from Example 1, Step 1 (6 g) was dissolved in 100 mL of ethanol saturated with ammonia. The reaction mixture was allowed to stand two days at room temperature. The removal of excess ammonia and ethanol gave the intermediate amidol (5.8 g), which was dissolved in 100 mL of acetic acid. The hydrogenation was carried out in the presence of 5.5 g of Pd—C (5%, dry) at 80-100° C. for 1 hour. Hydrogenation was continued for another two hours at 55 psi hydrogen pressure. The reaction product was isolated after filtration and evaporation. Trituration with methyl t-butyl ether (MTBE) followed by ether gave 1.7 g of the desired lactam 6. ¹H NMR (CDCl₃) δ 6.6-6.4 (bs, 1H), 3.15 (s, 2H), 2.18 (s, 2H), 1.80-1.30 (m, 10H) ¹³C NMR (CDCl₃) d 178.6, 54.0, 43.5, 39.6, 37.0, 25.9, 23.1.

Step 2: Preparation of gabapentin hydrochloride. The lactam (23 g, prepared according to Step 1) and concentrated hydrochloric acid (500 mL) are heated under reflux for 16 hours. Water and excess HCl are removed by evaporation in vacuo to give crude gabapentin hydrochloride, which is purified by crystallization from butanol/ether.

### EXAMPLE 4

### Synthesis of Gabapentin by Method A

Step 1: Preparation of benzyl (1-formylcyclohexyl)acetate. A mixture of 5.6 g of cyclohexane carboxaldehyde and diisobutyl amine (7 g) was refluxed in toluene (150 mL). After removal of approximately 0.5 mL of water, toluene was removed by distillation. Acetonitrile (60 mL) was added followed by the addition of 4.58 g of benzyl 2-bromoacetate. The reaction mixture was refluxed for 16 hours. Acetic acid (5 mL) and 25 mL of water were added, and the mixture heated for additional 2 hours. The solvents were evaporated and the product was extracted with ether. Five grams of the crude material was purified by column chromatography (silica) to give 1.5 g of benzyl (1-formylcyclohexyl)acetate. NMR (CDCl₃) δ 9.67 (s, 1H), 7.45-7.25 (m, 5H), 5.10 (s, 2H), 2.63 (s, 2H), 1.90-1.70 (m, 2H); 1.60-1.40 (m, 8H).

Step 2: Preparation of gabapentin hydrochloride. Benzyl (1-formylcyclohexyl)-acetate (7.8 g) was taken up in 300 mL of ethanol saturated with ammonia. The mixture was hydrogenated with Pd—C (10%, 0.8 g) for 6 hours under 50 psi hydrogen pressure. The catalyst was filtered off and the solvent evaporated to give crude gabapentin, which was purified by crystallization from ethanol/ether.

### EXAMPLE 5

### Synthesis of Gabapentin by Method A

Step 1: Preparation of ethyl (1-hydroxyiminomethylcyclohexyl)acetate. A mixture of cyclohexanecarboxaldehyde (22.4 g) and diisobutyl amine (28 g) in toluene (150 mL) was heated with azeotropic removal of about 3.5 mL of water. Toluene was removed by evaporation in vacuo and the residue was treated with 200 mL of acetonitrile. Ethyl bromoacetate (40 g) was added and the reaction was heated under reflux for 16 hours. A solution of hydroxylamine hydrochloride (13.9 g) in 100 mL of water was added. A 10% solution of sodium carbonate was added to adjust the pH of the reaction to 8.0. The reaction mixture was allowed to stand at ambient temperature for 16 hours. The reaction mixture was distilled to reduce the volume by ⅔. Water (200 mL) was added and the product extracted with ether. Distillation of the solvent and the diisopropyl amine gave the oxime, ethyl (1-hydroxyiminomethylcyclohexyl)acetate.

Step 2: Preparation of gabapentin hydrochloride. A solution of the oxime prepared in Step 1 (2.15 g) was dissolved in acetic acid, and hydrogenated at 80° C., 50 psi using 5% Pd—C (4.0 g) for 6 hours. The reaction mixture was filtered and evaporated in vacuo. The residue was treated with 60 mL of 50% concentrated HCl and heated under reflux for 14 hours. Thereafter, the solution was co-evaporated with 50 mL of n-butanol. Trituration with THF and MTBE gave 0.67 g of gabapentin hydrochloride.

### EXAMPLE 6

### Synthesis of Gabapentin by Method B

Step 1: Preparation of the oxime, ethyl (1-hydroxyiminomethylcyclohexyl)acetate. A solution of hydroxylamine hydrochloride (1.4 g) in water (10 mL) was added to a solution of ethyl(1-formylcyclohexyl)acetate from Step 1, Example 1, in ethanol (20 mL). The reaction was made alkaline (pH 8) using 10% sodium carbonate solution. After stirring for 16 hours at room temperature, the solution was evaporated in vacuo, and the oxime was isolated by extraction with ether. Yield 3.95 g. ¹H NMR (CDCl₃) d 7.9 (bs, 1H), 7.44 (s, 1H), 4.11 (q, 2H, J=7.1 Hz), 2.44 (s, 2H), 1.90-1.75 (m, 2H), 1.60-1.40 (m, 8H), 1.24 (t, 3H, J=7.1 Hz).

Step 2: The reduction and hydrolysis of ethyl (1-hydroxyiminomethylcyclohexyl)-acetate to gabapentin hydrochloride was carried out as described in Step 2, Example 5.

### EXAMPLE 7

### Synthesis of Gabapentin by Method B

Step 1: Preparation of benzyl (1-hydroxyiminomethylcyclohexyl)acetate. A solution of hydroxylamine hydrochloride (2.8 g) in water (20 mL) was added to a solution of benzyl (1-formylcyclohexyl)acetate from Step 1, Example 4, in ethanol (40 mL). The reaction was made alkaline (pH 8) using 10% sodium carbonate solution. After stirring for 16 hours at room temperature, the solution was evaporated in vacuo, and the benzyl (1-hydroxyiminomethylcyclohexyl)acetate was isolated by extraction with ether.

Step 2: Preparation of gabapentin hydrochloride. A solution of the oxime prepared in Step 1 (8.26 g) was dissolved in acetic acid, and hydrogenated at 80° C., 50 psi using of 5% Pd—C (4.0 g) for 6 hours. Further work-up was carried out according to Step 2, Example 3.

### EXAMPLE 8

### Synthesis of Gabapentin by Method B

Step 1: Preparation of [1-(hydroxyiminomethyl)cyclohexyl]acetic acid. (1-Formyl-cyclohexyl)acetic acid (17 g) from Step 1, Example 2 and hydroxylamine hydrochloride (7.0 g) is taken in 200 mL of water. A solution of sodium carbonate (10%) was added to adjust the pH to 8.0. After stirring for 16 hours at room temperature the product was extracted with ether.

Step 2: Preparation of gabapentin hydrochloride. A solution of [1-(hydroxyimino-methyl)cyclohexyl]acetic acid (9.26 g) prepared according to Step 1, was dissolved in acetic acid, and hydrogenated at 80° C., 50 psi using 5% Pd—C (1.0 g) for 6 hours. The reaction mixture was filtered and evaporated in vacuo. The solid residue was washed with ethanol. The combined ethanol solution was concentrated and gabapentin was purified by crystallization from ethanol and ether.

1. A process for the preparation of gabapentin which comprises the steps of:
(a) reacting a carboxaldehyde selected from the group consisting of cyclohexanecarboxaldehyde and cyclohexenecarboxaldehyde with an amine selected from the group consisting of secondary alkyl and arylalkyl amines;
(b) reacting the resultant enamine with an alkylating agent having the formula Y—CH₂—X, wherein Y is a leaving group selected from halogen, C₁-C₁₀ alkane sulfonate, and C₅-C₁₀ arene sulfonate and X is selected from the group consisting of —CN, —CO₂M, —CO₂R₃ and —CONR₄R₅, with R₃ to R₅ being independently selected from the group consisting of hydrogen, cyanoethyl, alkyl cycloalkyl, aryl unsubstituted or substituted with electron withdrawing or electron donating groups; arylalkyl unsubstituted or substituted with electron withdrawing or electron donating groups, and M is selected from the group consisting of lithium, sodium, potassium, calcium, magnesium, trialkylammonium and tetralkylammonium;
(c) converting the resultant iminium salt to gabapentin.

2. A method as in claim 1 wherein R¹ and R² are benzyl groups and the conversion of Step (c) to produce gabapentin is accomplished by direct reductive amination.

3. A method as in claim 1 wherein the conversion of Step (c) is accomplished by hydrolysis to an aldehyde followed by reduction to gabapentin.

4. A method as in claim 1 in which Step (c) comprises hydrolysis of the iminium salt to an aldehyde wherein X is a benzyl ester, acid or a salt and the conversion to gabapentin is accomplished by direct reductive amination.

5. A method as in claim 1 in which Step (c) comprises hydrolysis of the iminium salt to an aldehyde wherein X is other than a benzyl ester, acid or a salt, followed by amination to form the lactam and hydrogenolysis to produce gabapentin.

6. A process for the preparation of gabapentin which comprises:
(a) reacting diisobutyl amine and cyclohexanecarboxaldehyde to produce cyclohexylidenemethyl-diisobutyl amine;
(b) alkylating said cyclohexylidenemethyl-diisobutyl amine by reaction with ethylbromoacetate to produce (1-ethoxycarbonylmethyl-cyclohexylmethylene)-diisobutyl ammonium bromide;
(c) hydrolyzing said (1-ethoxycarbonylmethyl-cyclohexylmethylene)-diisobutyl ammonium bromide to produce ethyl (1-formylcyclohexyl)acetate;
(d) subjecting said ethyl (1-formylcyclohexyl)acetate to direct reductive amination to produce gabapentin.
